(19) United States
(12) Patent Application Publication
(10) Pub. No.: US 2003/0069440 A1
(43) Pub. Date: Apr. 10, 2003

(54) Preparation of acrylic polyols

(76) Inventors: Shao-Hua Guo, Exton, PA (US); Wei Wang, Boothwyn, PA (US); Daniel B. Pourreau, Exton, PA (US)

Correspondence Address: LYONDELL CHEMICAL COMPANY, 3801 WEST CHESTER PIKE, NEWTOWN SQUARE, PA, 19073, US

(21) Appl. No.: 09/934,878

(22) Filed: Aug. 22, 2001

(51) Int. Cl. C07C 69/52

(52) U.S. Cl. 560/224

(57) ABSTRACT

A process for making acrylic polyols from allylic alcohols is disclosed. The process is performed essentially in the absence of styrene and in the absence of methyl acrylate or methacrylate. It comprises initially charging a reactor with an allylic alcohol, 0-75% of the total amount to be used of an acrylic monomer, and 0-100% of the total amount to be used of a free-radical initiator. The reaction mixture is heated to a temperature within the range of about 100° C. to about 250° C. The remaining acrylic monomer and initiator are gradually added into the reactor during the course of polymerization. The monomer conversion is greatly enhanced as a result of using essentially no styrene.

### FIELD OF THE INVENTION

The invention relates to a process for making acrylic polyols. More particularly, the invention relates to a process for making acrylic polyols from allylic alcohols. The process gives a high monomer conversion as a result of using essentially no styrene.

### BACKGROUND OF THE INVENTION

Acrylic polyols have been widely and increasingly used in high performance coatings, particularly in automotive topcoats, due to their excellent durability and outstanding physical properties. They are usually crosslinked with a multifunctional isocyanate or a melamine to form acrylic-urethane or acrylic-melamine coatings.

Acrylic polyols are usually copolymers of a hydroxyalkyl acrylate or methacrylate and one or more alkyl acrylates or methacrylates. Commonly used hydroxyalkyl acrylates and methacrylates include hydroxyethyl acrylate (HEA), hydroxypropyl acrylate (HPA), hydroxyethyl methacrylate (HEMA), and hydroxypropyl methacrylate (HPMA).

Usually, a combination of two types of alkyl acrylates and methacrylates is used to achieve optimal resin properties. The first type includes acrylates and methacrylates whose homopolymers have low glass transition temperatures (T_{g} below 25° C.), e.g., n-butyl acrylate (T_{g}: −54° C.), n-butyl methacrylate (T_{g}: 20° C.), and 2-ethylhexyl methacrylate (T_{g}: 10° C.). The second type includes acrylates and methacrylates whose homopolymers have high T_{g}s (greater than 50° C.), such as methyl methacrylate (T_{g}: 100° C.). Styrene is also often incorporated into acrylic resins as a high-T_{g} monomer (T_{g}: 99° C.). High-T_{g} monomers increase the coating's gloss and hardness, while low-T_{g} monomers impart toughness and flexibility.

In general, high-solids acrylic resins have a hydroxyl number from 60 to 160 mg KOH/g, and a number average molecular weight (Mn) from 1,000 to 5,000. Lowering the molecular weight of the acrylic polyol can reduce its solution viscosity. This is desirable because it reduces the amount of solvent required to make the coatings sprayable. Solvents are regulated as volatile organic compounds (VOCs) by the U.S. EPA and most coatings have VOC content limits imposed on them. However, the molecular weight reduction must be compensated by an increase in resin hydroxyl number to maintain the coating performance. The increased hydroxyl number increases hydrogen bonding within the resin which increases viscosity. The current solids level of sprayable acrylic-urethane or acrylic-melamine coatings is about 50% to 55% by weight.

Newly developed acrylic polyols from allylic alcohols have significantly reduced viscosity, and their melamine and urethane coatings can achieve about 60% solids (see, e.g., U.S. Pat. No. 5,646,213). However, making acrylic polyols from allylic monomers is difficult because of low monomer conversion. Removing and recycling unreacted monomers is inconvenient and costly. Thus, a process that gives high monomer conversion is needed.

### SUMMARY OF THE INVENTION

The invention is a process for making acrylic polyols from allylic alcohols. The process is performed essentially in the absence of styrene and in the absence of methyl acrylate or methacrylate. It comprises initially charging a reactor with an allylic alcohol, 0-75% of the total amount to be used of a C₂ to C₂₀ alkyl or aryl acrylate or methacrylate, and 0-100% of the total amount to be used of a free-radical initiator. The reaction mixture is heated to a temperature within the range of 100-250° C. The remaining acrylic monomer and initiator are gradually added into the reactor during the course of polymerization. The process gives a high monomer conversion as a result of using essentially no styrene and no methyl acrylate or methacrylate.

### DETAILED DESCRIPTION OF THE INVENTION

The process of the invention comprises initially charging a reactor with an allylic alcohol, 0-75% of the total amount to be used of a C₂ to C₂₀ alkyl or aryl acrylate or methacrylate, and 0-100% of the total amount to be used of a free-radical initiator. The reaction mixture is heated to a temperature within the range of about 100° C. to about 250° C. The remaining acrylic monomer and initiator are gradually added into the reactor during the course of polymerization.

The process is performed essentially in the absence of styrene. “By essentially in the absence of styrene,” we mean that the styrene is less than 5% of the amount of the acrylic monomer used. Preferably, styrene is used in an amount less than 1% of the acrylic monomer. As a result of using essentially no styrene, the total monomer conversion is significantly enhanced. By “total monomer conversion,” we mean the ratio of the amount of acrylic polyol produced over the total amount of monomers used, i.e., the amount of allylic alcohol plus the amount of acrylic monomer. Preferably, the total monomer conversion is greater than about 90%. More preferably, the total monomer conversion is greater than about 95%. Most preferably, the total monomer conversion is greater than about 99%.

Although allylic alcohols are known to be useful hydroxyl functional monomers for making acrylic polyols, their use is limited because they give a low monomer conversion. Removing and recycling or disposing of unreacted monomers are costly and inconvenient. While styrene is commonly used in acrylic polyols, we have surprisingly found that using essentially no styrene in the process significantly enhances the total monomer conversion. For instance, when styrene is used in the copolymerization of allyl alcohol monopropoxylate and n-butyl acrylate, the total monomer conversion is only 88.8% (Comparative Example 3). With no styrene presence, the total monomer conversion is 99.5% (Example 1).

When the total monomer conversion is greater than about 90%, not only is the productivity of the process significantly increased, but also the costs for removing and recycling the unreacted monomers are reduced. Furthermore, when the total monomer conversion is greater than about 99%, removing and recycling the unreacted monomer may no longer be necessary.

Preferably, the allylic alcohols have the general structure:

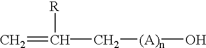

in which R is selected from the group consisting of hydrogen, a C₁-C₁₀ alkyl group, and a C₆-C₁₂ aryl group. Preferably, R is hydrogen or a methyl group. A is an oxyalkylene group. Preferably, A is selected from the group consisting of oxyethylene, oxypropylene, oxybutene, and mixtures thereof. Preferably, n is an average number of oxyalkylene units, which is within the range of 0 to about 15. More preferably, n is within the range of about 1 to about 5. Most preferably, n is from about 1 to about 2. Examples of allylic alcohols include allyl alcohol, methallyl alcohol, allyl alcohol monopropoxylate, allyl alcohol monoethoxylate, methallyl alcohol monopropoxylate, allyl alcohol propoxylate having an average 1.6 oxypropylene units, the like, and mixtures thereof. Allyl alcohol monopropoxylate is particularly preferred.

Acrylic monomers suitable for the use in the process of the invention include C₂ to C₂₀ alkyl and aryl acrylates and methacrylates. C₂ to C₂₀ alkyl acrylates and methacrylates are preferred. Examples of suitable acrylic monomers are ethyl acrylate, 2-ethylbutyl acrylate, 2-ethylhexyl acrylate, n-octyl acrylate, 2-octyl acrylate, propyl acrylate, n-butyl acrylate, sec-butyl acrylate, lauryl acrylate, decyl methacrylate, 2-ethylhexyl methacrylate, hexyl methacrylate, n-octyl methacrylate, lauryl methacrylate, n-butyl methacrylate, t-butyl methacrylate, isobornyl methacrylate, and the like, and mixtures thereof.

The ratio of allylic alcohol/acrylic monomer is preferably within the range of about 10/90 to about 90/10 by weight. More preferably, the ratio is from 10/90 to 50/50.

Methyl acrylate and methyl methacrylate are not suitable for use in the process of the invention because they undergo a trans-esterification reaction with allylic alcohols (particularly allyl alcohol) to form six-membered lactones. See S. H. Guo, Solvent-Free Polymerizations and Processes, ACS Series Book, No. 713, Chapter 7, pp. 113-126 (1998). The lactone formation reduces the hydroxyl number of the resulting acrylic polyol, causes gel formation, and decreases the total monomer conversion.

Suitable free-radical initiators include peroxides, hydroperoxides, azo compounds, and many others known to the polymer industry. Examples of suitable free-radical initiators are hydrogen peroxide, di-t-butyl peroxide, t-butyl hydroperoxide, cumene hydroperoxide, 2,2′-azobisisobutyronitrile, and the like, and mixtures thereof.

0-75% of the amount to be used of the acrylic monomer is initially charged into the reactor. The remaining acrylic monomer is gradually added into the reactor during the course of polymerization. Preferably, the acrylic monomer is added at a decreasing rate to keep the ratio of acrylic monomer to allylic alcohol essentially constant in the reaction mixture so that the resin produced has a relatively uniform composition.

The free-radical initiator can be initially added into the reactor. However, it is preferred to add at least 50% of the total amount to be used of the initiator to the reactor gradually during the course of the polymerization. It is also desirable to match the addition rate of the free-radical initiator to the addition rate of the acrylic monomer, so that the resin produced has a uniform composition and a narrow molecular weight distribution. Gradual addition of the initiator can also increase the monomer conversion.

The polymerization is conducted at a temperature within the range of about 100° C. to about 280° C. More preferably, the temperature is within the range of about 125° C. to about 165° C. The polymerization can be performed in a closed reactor under pressure. Closed-reactor polymerization is particularly preferred when allyl alcohol is used because of its low boiling point and high toxicity. When the polymerization is performed in a closed reactor, the reaction heat is removed through a reactor jacket or an internal coil. The reaction temperature can be kept essentially constant through the course of polymerization. Alternatively, the reaction temperature can gradually increase as the polymerization continues. U.S. Pat. No. 6,103,840, the teachings of which are herein incorporated by reference, teaches how to program a temperature increase to enhance the polymer yield.

Alternatively, polymerization is performed under atmospheric pressure and at the reflux temperature of the reaction mixture. When the polymerization is so performed, the allylic alcohol preferably has a lower boiling point than the acrylic monomer, so that the concentration of allylic alcohol in the vapor phase is higher than the acrylic monomer. Allylic alcohols, although they readily copolymerize with acrylic monomers, do not homopolymerize rapidly. On the other hand, acrylic monomers undergo rapid homopolymerization and oxidation. Thus, it is preferred to keep a low concentration of acrylic monomer in the vapor phase. In addition, the allylic alcohol used preferably has a high boiling point (greater than about 100° C.) so that the polymerization is performed at a high reflux temperature.

Similarly, the free-radical initiator preferably has a higher boiling point than the allylic alcohol in the polymerization under reflux so that the initiator remains in the liquid phase where the polymerization occurs. Preferably, the initiator does not contain a high concentration of low boiling point solvent that may lower the reflux temperature and thus reduce the monomer conversion. For instance, using di-t-butyl peroxide gives 93.7% of the total monomer conversion in the atmospheric pressure polymerization of allyl alcohol monopropoxylate and n-butyl acrylate (see Example 8). However, T-hydro® 70 (product of Lyondell Chemical Company), which is a 70% solution of t-butyl hydroperoxide in water, gives only 86% of the total monomer conversion (Comparative Example 10). The high water content of T-hydro 70 lowers the reflux temperature and thus reduces the monomer conversion.

Preferably, the acrylic polyols produced have a number average molecular weight (Mn) less than about 5,000, a molecular weight distribution less than about 3.5, and a hydroxyl number within the range of about 20 mg KOH/g to about 500 mg KOH/g. More preferably, the acrylic polyol has Mn less than 3,000 and a hydroxyl number within the range of 75 mg KOH/g to 150 mg KOH/g. The acrylic polyols made by the process of the invention are known to have a more even distribution of the hydroxyl functional groups than conventional acrylic polyols made from hydroxyalkyl acrylates or methacrylates because allylic alcohols do not homopolymerize rapidly.

The process of the invention is particularly suitable for making liquid acrylic polyols because liquid acrylic polyols usually do not contain recurring units of styrene. Co-pending application Ser. No. 09/391,562, the teachings of which are incorporated herein by reference, teaches using a blend of a liquid acrylic polyol and a resinous polyol to formulate ultra-high solids coatings. Preferably, the liquid acrylic polyol has a T_{g} within the range of about −70° C. to about 0° C. Acrylic monomers suitable for making liquid acrylic polyols are those which have a homopolymer T_{g} below about 0° C. Examples are ethyl acrylate, 2-ethylbutyl acrylate, 2-ethylhexyl acrylate, n-octyl acrylate, 2-octyl acrylate, propyl acrylate, n-butyl acrylate, sec-butyl acrylate, lauryl acrylate, decyl methacrylate, 2-ethylhexyl methacrylate, hexyl methacrylate, n-octyl methacrylate, lauryl methacrylate, and the like, and mixtures thereof.

The following examples merely illustrate the invention. Those skilled in the art will recognize many variations that are within the spirit of the invention and scope of the claims.

### EXAMPLE 1

### Preparation of Allyl Alcohol Monopropoxylate/n-Butyl Acrylate Copolymer under Pressure

A five-liter stainless-steel reactor equipped with an agitator, oil heating jacket, temperature controller, nitrogen purge device, vacuum distillation device, and pumps for monomers or initiator, is charged with allyl alcohol monopropoxylate (655 g). n-Butyl acrylate (1410 g) is purged with nitrogen and charged to the monomer addition pump. T-hydro 70 (205 g, t-butyl hydroperoxide, 70% aqueous solution, product of Lyondell Chemical) is purged with nitrogen and charged to the initiator addition pump. The reactor is purged three times with nitrogen, sealed, and the reactor contents are heated to 145° C. Butyl acrylate and initiator are added to the reactor gradually at a decreasing rate over 6 hours while maintaining the reaction temperature at 145° C. The addition rate of butyl acrylate is: hour 1: 300 g; hour 2: 285 g; hour 3: 250 g; hour 4: 225 g; hour 5: 200 g; and hour 6: 150 g. The addition rate of the initiator is hour 1: 44 g; hour 2: 41 g; hour 3: 36 g; hour 4: 33 g; hour 5: 29 g; and hour 6: 22 g. The reaction mixture is kept at 145° C. for another 0.5 hour following monomer and initiator addition. Unreacted monomers are removed by vacuum distillation at 150° C. The acrylic polyol (2054 g) is discharged from the reactor at 50° C.; the total monomer conversion is 99.5%. It is a liquid at 25° C. and has a composition: 25% allyl alcohol monopropoxylate units and 75% butyl acrylate units, number average molecular weight (Mn): 2,830, molecular weight distribution (Mw/Mn): 2.47, hydroxyl (OH) number: 121 mg KOH/g, Brookfield viscosity at 25° C.: 23,600 cps, and T_{g}: −48° C.

### EXAMPLE 2

### Preparation of Allyl Alcohol Monopropoxylate/2-Ethylhexyl Acrylate Copolymer under Pressure

A reactor equipped as in Example 1 is charged with allyl alcohol monopropoxylate (730 g), 2-ethylhexyl acrylate (185 g), and T-hydro 70 (43 g). Additional 2-ethylhexyl acrylate (1250 g) is purged with nitrogen and charged to the monomer addition pump. Additional T-hydro 70 (147 g) is purged with nitrogen and charged to the initiator addition pump. The reactor is purged three times with nitrogen, sealed, and the contents are heated to 145° C. 2-Ethylhexyl acrylate and initiator are added to the reactor gradually at a decreasing rate over 5 hours while maintaining the reaction temperature at 145° C. The addition rate of 2-ethylhexyl acrylate is: hour 1: 330 g; hour 2: 315 g; hour 3: 275 g; hour 4: 220 g; and hour 5: 110 g. The addition rate of the initiator is hour 1: 39 g; hour 2: 37 g; hour 3: 32 g; hour 4: 26 g; and hour 5: 13 g. The reaction mixture is kept at 145° C. for another 0.5 hour following monomer and initiator addition. Unreacted monomers are removed by vacuum distillation at 155° C. The acrylic polyol (2103 g) is discharged from the reactor at 50° C.; the total monomer conversion is 97.1%. It is a liquid at 25° C. and has a composition: 26 wt % allyl alcohol monopropoxylate and 74 wt % 2-ethylhexyl acrylate, Mn: 2,340, Mw/Mn: 2.0, OH number: 130 mg KOH/g, Brookfield viscosity at 25° C.: 22,600 cps, and T_{g}: −48° C.

### COMPARATIVE EXAMPLE 3

### Preparation of Allyl Alcohol Monopropoxylate/n-Butyl Acrylate/Styrene Terpolymer under Pressure

A reactor equipped as in Example 1 is charged with allyl alcohol monopropoxylate (670 g). n-Butyl acrylate (1075 g) and styrene (350 g) are mixed, purged with nitrogen, and charged to the monomer addition pump. T-hydro 70 (210 g) is purged with nitrogen and charged to the initiator addition pump. The reactor is purged three times with nitrogen, sealed, and the contents are heated to 145° C. Monomer mixture and initiator are added to the reactor gradually at a decreasing rate over 6 hours while maintaining the reaction temperature at 145° C. The addition rate of the monomer mixture is hour 1: 315 g; hour 2: 285 g; hour 3: 250 g; hour 4: 225 g; hour 5: 200 g; and hour 6: 150 g. The addition rate of the initiator is hour 1: 46 g; hour 2: 42 g; hour 3: 37 g; hour 4: 33 g; hour 5: 29 g; and hour 6: 23 g. The reaction mixture is kept at 145° C. for another 0.5 hour following monomer and initiator addition. Unreacted monomers are removed by vacuum distillation at 155° C. The acrylic polyol (1859.4 g) is discharged from the reactor at 50° C.; the total monomer conversion is 88.8%. It has a composition: 25 wt % allyl alcohol monopropoxylate, 57 wt % n-butyl acrylate, and 18 wt % styrene, Mn: 2400, Mw/Mn: 2.86, OH number: 125 mg KOH/g, and T_{g}: −23° C.

### EXAMPLE 4

### Preparation of Allyl Alcohol Monopropoxylate/n-Butyl Methacrylate Copolymer under Pressure

A reactor equipped as in Example 1 is charged with allyl alcohol monopropoxylate (670 g). n-Butyl methacrylate (1410 g) is purged with nitrogen and charged to the monomer addition pump. T-hydro 70 (210 g) is purged with nitrogen and charged to the initiator addition pump. The reactor is purged three times with nitrogen, sealed, and the contents are heated to 145° C. n-Butyl methacrylate and initiator are added to the reactor gradually at a decreasing rate over 6 hours while maintaining the reaction temperature at 145° C. The addition rate of n-butyl methacrylate is hour 1: 300 g; hour 2: 285 g; hour 3: 250 g; hour 4: 225 g; hour 5: 200 g; and hour 6: 150 g. The addition rate of the initiator is hour 1: 46 g; hour 2: 42 g; hour 3: 37 g; hour 4: 33 g; hour 5: 29 g; and hour 6: 23 g. The reaction mixture is kept at 145° C. for another 0.5 hour following monomer and initiator addition. Unreacted monomers are removed by vacuum distillation at 155° C. The acrylic polyol (2021 g) is discharged from the reactor at 50° C.; the total monomer conversion is 97.2%. It has a composition: 25 wt % allyl alcohol monopropoxylate and 75 wt % n-butyl methacrylate, Mn: 2280, Mw/Mn: 2.23, OH number: 125 mg KOH/g, and T_{g}: −22° C.

### COMPARATIVE EXAMPLE 5

### Preparation of Allyl Alcohol Monopropoxylate/n-Butyl Methacrylate/Styrene Terpolymer under Pressure

A reactor equipped as in Example 1 is charged with allyl alcohol monopropoxylate (670 g). n-Butyl methacrylate (1075 g) and styrene (350 g) are mixed, purged with nitrogen, and charged to the monomer addition pump. T-hydro 70 (210 g) is purged with nitrogen and charged to the initiator addition pump. The reactor is purged three times with nitrogen, sealed, and the contents are heated to 145° C. Monomer mixture and initiator are added to the reactor gradually at a decreasing rate over 6 hours while maintaining the reaction temperature at 145° C. The addition rate of monomers is hour 1: 315 g; hour 2: 285 g; hour 3: 250 g; hour 4: 225 g; hour 5: 200 g; and hour 6: 150 g. The addition rate of the initiator is hour 1: 46 g; hour 2: 42 g; hour 3: 37 g; hour 4: 33 g; hour 5: 29 g; and hour 6: 23 g. The reaction mixture is kept at 145° C. for another 0.5 hour following monomer and initiator addition. Unreacted monomers are removed by vacuum distillation at 155° C. The acrylic polyol (1859 g) is discharged from the reactor at 50° C.; the total monomer conversion is 88.7%. It has a composition: 23 wt % allyl alcohol monopropoxylate, 58 wt % n-butyl methacrylate, and 19 wt % styrene, Mn: 2010, Mw/Mn: 2.33, OH number: 112.5 mg KOH/g, and T_{g}: −11° C.

### EXAMPLE 6

### Preparation of Allyl Alcohol Monopropoxylate/Isobornyl Methacrylate Copolymer under Pressure

A reactor equipped as in Example 1 is charged with allyl alcohol monopropoxylate (725 g). Isobornyl methacrylate (1545 g) is purged with nitrogen and charged to the monomer addition pump. T-hydro 70 (183 g) is purged with nitrogen and charged to the initiator addition pump. The reactor is purged three times with nitrogen, sealed, and the contents are heated to 145° C. Isobornyl methacrylate and initiator are added to the reactor gradually at a decreasing rate over 6 hours while maintaining the reaction temperature at 145° C. The addition rate of isobornyl methacrylate is hour 1: 330 g; hour 2: 310 g; hour 3: 275 g; hour 4: 250 g; hour 5: 215 g; and hour 6: 165 g. The addition rate of the initiator is hour 1: 39 g; hour 2: 36.5 g; hour 3: 32.5 g; hour 4: 29.5 g; hour 5: 25.5 g; and hour 6: 20 g. The reaction mixture is kept at 145° C. for another 0.5 hour following monomer and initiator addition. Unreacted monomers are removed by vacuum distillation at 155° C. The acrylic polyol (2248.4 g) is discharged from the reactor at 50° C.; the total monomer conversion is 99.0%. It has a composition: 24 wt % allyl alcohol monopropoxylate and 76 wt % isobornyl methacrylate, Mn: 1450, MwlMn: 2.14, OH number: 115 mg KOH/g, and T_{g}: 34° C.

### COMPARATIVE EXAMPLE 7

### Preparation of Allyl Alcohol Monopropoxylate/Isobornyl Methacrylate/Styrene Terpolymer under Pressure

A reactor equipped as in Example 1 is charged with allyl alcohol monopropoxylate (725 g). Isobornyl methacrylate (1170 g) and styrene (375 g) are mixed, purged with nitrogen, and charged to the monomer addition pump. T-hydro 70 (183 g) is purged with nitrogen and charged to the initiator addition pump. The reactor is purged three times with nitrogen, sealed, and the contents are heated to 145° C. The monomers and initiator are added to the reactor gradually at a decreasing rate over 6 hours while maintaining the reaction temperature at 145° C. The addition rate of the monomer mixture is hour 1: 330 g; hour 2: 310 g; hour 3: 280 g; hour 4: 250 g; hour 5: 215 g; and hour 6: 160 g. The addition rate of the initiator is hour 1: 48 g; hour 2: 43 g; hour 3: 36 g; hour 4: 29 g; hour 5: 23 g; and hour 6: 16 g. The reaction mixture is kept at 145° C. for another 0.5 hour following monomer and initiator addition. Unreacted monomers are removed by vacuum distillation at 155° C. The acrylic polyol (1990.5) is discharged from the reactor at 50° C.; the total monomer conversion is 87.7%. It has composition: 25 wt % allyl alcohol monopropoxylate, 57 wt % isobornyl methacrylate, and 18 wt % styrene, Mn: 1910, Mw/Mn: 2.57, OH number: 120 mg KOH/g, and T_{g}: 44° C.

TABLE 1
The Effect of Styrene on Monomer Conversion

| Example No | 1 | 2 | C3 | 4 | C5 | 6 | C7 |
|---|---|---|---|---|---|---|---|
| Presence of | No | No | Yes | No | Yes | No | Yes |
| Styrene | | | | | | | |
| Monomer | 99.5 | 97.1 | 88.8 | 97.2 | 88.7 | 99.0 | 87.7 |
| Conversion % | | | | | | | |

### EXAMPLE 8

### Preparation of Allyl Alcohol Monopropoxylate/n-Butyl Acrylate Copolymer at Atmospheric Pressure with Di-t-Butyl Peroxide

A two-liter glass reactor equipped with an agitator, heating mantle, monomer/initiator addition pump, nitrogen inlet, and reflux condenser is charged with allyl alcohol monopropoxylate (365 g). n-Butyl acrylate (710 g) and di-t-butyl peroxide (80 g) are mixed, purged with nitrogen, and charged to the addition pump. The reactor is purged three times with nitrogen and the contents are heated to the reflux temperature (145° C.). The mixture of monomer and initiator is added to the reactor over 6 hours at a decreasing rate while maintaining the reactor contents at reflux (145° C.). The addition rate is hour 1: 193 g; hour 2: 169 g; hour 3: 145 g; hour 4: 120 g; hour 5: 98 g; and hour 6: 65 g. The reaction mixture is kept refluxing (145° C.) for another 0.5 hour following the addition. Unreacted monomers are removed by vacuum distillation at 155° C. The acrylic polyol (1007 g) is collected; the total monomer conversion is 93.7%. It is a liquid at 25° C. and has Mn: 2470, Mw/Mn: 3.41, OH number: 130 mg KOH/g, T_{g}: 45° C., and Brookfield viscosity: 51,700 cps.

### EXAMPLE 9

### Preparation of Allyl Alcohol Monopropoxylate/n-Butyl Acrylate Copolymer at Atmospheric Pressure with T-hydro 90

A reactor equipped as in Example 8 is charged with allyl alcohol monopropoxylate (365 g), n-butyl acrylate (90 g), and T-hydro 90 (18 g, 90% aqueous solution of t-butyl hydroperoxide, product of Aldrich). The reactor is purged three times with nitrogen and the contents are heated to the reflux temperature (145° C.). n-Butyl acrylate (620 g) and T-hydro 90 (62 g) are mixed, purged with nitrogen, and charged to the addition pump. The mixture is added to the reactor over 5 hours at a decreasing rate under reflux temperature (145° C.). The addition rate is hour 1: 180 g; hour 2: 170 g; hour 3: 150 g; hour 4: 120 g; and hour 5: 62 g. The reaction mixture is kept refluxing (145° C.) for another 0.5 hour following the addition. Unreacted monomers are removed by vacuum distillation at 155° C. The acrylic polyol (1017 g) is collected; the total monomer conversion is 93.7%. It is a liquid at 25° C. and has Mn: 2,510, Mw/Mn: 2.40, OH number: 130 mg KOH/g, T_{g}: −45° C., and Brookfield viscosity: 39,400 cps.

### COMPARATIVE EXAMPLE 10

### Preparation of Allyl Alcohol Monopropoxylate/n-Butyl Acrylate Copolymer at Atmospheric Pressure with T-hydro 70

A reactor equipped as in Example 9 is charged with allyl alcohol monopropoxylate (295 g). n-Butyl acrylate (635 g) and T-hydro 70 (92 g) are mixed and charged into the addition pump. The reactor is purged three times with nitrogen and the contents are heated to reflux (145° C.). The monomer and initiator mixture is added into the reactor over 6 hours at a decreasing rate under reflux while maintaining the reactor contents at reflux. The addition rate is hour 1: 155 g; hour 2: 147 g; hour 3: 129 g; hour 4: 116 g; hour 5: 103 g; and hour 6: 77 g. The reflux temperature gradually drops from 145° C. to 118° C. during the addition. The reaction mixture is kept refluxing (118° C.) following the addition. The acrylic polyol (799.6 g) is collected; the total monomer conversion is 86.0%. It has Mn: 3,515, Mw/Mn: 3.54, OH number: 116 mg KOH/g, T_{g}: −40° C., and Brookfield viscosity: 95,500 cps.

### EXAMPLE 11

### Preparation of Allyl Alcohol Monopropoxylate/n-Hexyl Acrylate Copolymer at Atmospheric Pressure

The procedure of Example 8 is repeated, but n-butyl acrylate is replaced by n-hexyl acrylate. The acrylic polyol (1008 g) is collected; the total monomer conversion is 93.8%. It has Mn: 2,400, Mw/Mn: 2.63, OH number: 120 mg KOH/g, T_{g}: −48° C., and Brookfield viscosity: 19,700 cps.

### EXAMPLE 12

### Preparation of Allyl Alcohol Monopropoxylate/2-Ethylhexyl Acrylate Copolymer at Atmospheric Pressure

The procedure of Example 8 is repeated, but n-butyl acrylate is replaced by 2-ethylhexyl acrylate. The acrylic polyol (1022 g) is collected; the total monomer conversion is 95.1%. It has Mn: 2250, Mw/Mn: 2.55, OH number: 118 mg KOH/g, T_{g}: −52° C., and Brookfield viscosity: 30,800 cps.

### EXAMPLE 13

### Preparation of Allyl Alcohol Monopropoxylate/2-Ethylhexyl Acrylate Copolymer at Atmospheric Pressure

The procedure of Example 9, but n-butyl acrylate is replaced by 2-ethylhexyl acrylate. The acrylic polyol (1005 g) is collected; the total monomer conversion is 92.2%. It has Mn: 2330, Mw/Mn: 1.92, OH number: 122 mg KOH/g, T_{g}: −48° C., and Brookfield viscosity: 19,900 cps.

### EXAMPLE 14

### Preparation of Allyl Alcohol Monopropoxylate/2-Ethylhexyl Acrylate Copolymer at Atmospheric Pressure

A reactor equipped as in Example 8 is charged with allyl alcohol monopropoxylate (365 g), 2-ethylhexyl acrylate (94 g) and cumene hydroperoxide (14 g, 88% aqueous solution). Additional 2-ethylhexyl acrylate (631 g) and cumene hydroperoxide (51 g) are mixed, purged with nitrogen and charged into the addition pump. The reactor is purged three times with nitrogen and the contents are heated to reflux (145° C.). The monomer and initiator mixture is added into the reactor over 5 hours at reflux (145° C.). The addition rate is hour 1: 180 g; hour 2: 170 g; hour 3: 150 g; hour: 120 g; and hour 5: 62 g. The reaction mixture is keep refluxing for another 0.5 hour following the addition. Unreacted monomers are removed by vacuum distillation at 155° C. The acrylic polyol (1033 g) is collected; the i5 total monomer conversion is 94.8%. It has Mn: 2210, Mw/Mn: 1.88, OH number: 125 mg KOH/g, T_{g}: −51° C., and Brookfield viscosity: 15,680 cps.

### EXAMPLE 15

### Urethane-Acrylic Coating With 90/10 Blend of Liquid Acrylic Polyol/SAA-100 Resinous Polyol

The liquid acrylic resin of Example 1 (90 g) and SAA-100 resinous polyol (10 g, Tg: 62° C., hydroxyl number: 218 mg KOH/g, Mn: 1500, product of Lyondell Chemical Company) are dissolved in a mixture of xylene (32.5 g) and ethyl acetate (32.5 g). To this resin solution is added polymeric HDI (56.7 g, Luxate HT 2090, product of Lyondell Chemical Company) and dibutyltin dilaurate (0.38 g, 2% solution in methyl amyl ketone (MAK)). The solids content of the composition is 66.2% by weight and it has a viscosity of 95 centistokes at 25° C. The coating composition is drawn down on steel panels to a uniform wet film of thickness 3 mils with a Bird type film applicator. The panels are dried in a hood at 25° C. They are tested after five days and give the following results: Gloss at 20°: 87; Gloss at 60°: 100; Pencil Hardness: HB; Adhesion (ASM 3359): 5; Gardner Impact Direct: 144; Garner Impact Reverse: >160; Conical Mandrel Bend Test: 0.

### EXAMPLE 16

### Acrylic-Melamine Coating With 90/10 Blend of Liquid Acrylic Polyol/SAA-100 Resinous Polyol

The liquid acrylic polyol of Example 1 (63 g) and SAA-100 resinous polyol (7 g) are dissolved in MAK (47 g). To this resin solution is added Cymel 303 melamine resin (30 g, product of Cytec Chemical Company), Cycat 600 (1.0 g, p-toluenesulfonic acid, product of Cytec) and Dow 57(0.05 g, silicon deformer, product of Dow Chemical). The solids content of the composition is 68.3% by weight. Viscosity of the composition is 95 centistokes at 25° C. The coating is drawn down on steel panels to a uniform wet film of thickness 3 mils with a Bird type film applicator. The panels are air-dried in a hood for 30 min, and then baked in an oven at 80° C. for 30 minutes. The panels are tested after four days and give the following results: Gloss at 20°: 90; Gloss at 600: 101; Pencil Hardness: B; Adhesion (ASM 3359): 2; Gardner Impact Direct: 128; Gardner Impact reverse: 88; Conical Mandrel Bend Test: 0.

We claim:

1. A process for making an acrylic polyol, said process being performed essentially in the absence of styrene and in the absence of methyl acrylate and methyl methacrylate, and comprising:
(a) charging a reactor with an allylic alcohol, 0-75% of the total amount to be used of a C₂-C₂₀ alkyl or aryl acrylate or methacrylate monomer and 0-100% of the total amount to be used of a free-radical initiator;
(b) heating the reactor contents to a temperature within the range of 100-250° C.; and
(c) gradually adding to the reactor the remaining acrylic monomer and initiator.

2. The process of claim 1 giving a total monomer conversion greater than about 90%.

3. The process of claim 1 giving a total monomer conversion greater than about 95%.

4. The process of claim 1 giving a total monomer conversion greater than about 99%.

5. The process of claim 1 wherein the acrylic polyol has a number average molecular weight less than about 5,000 and a weight average molecular weight less than about 10,000.

6. The process of claim 1 wherein the acrylic polyol has a number average molecular weight less than about 2,500 and a weight average molecular weight less than about 5,000.

7. The process of claim 1 wherein the acrylic polyol has a molecular weight distribution less than about 3.5.

8. The process of claim 1 wherein the acrylic polyol has a molecular weight distribution less than about 2.5.

9. The process of claim 1 wherein the liquid acrylic polyol has a hydroxyl number within the range of about 20 mg KOH/g to about 250 mg KOH/g.

10. The process of claim 1 wherein the acrylic monomer is a C₂-C₂₀ alkyl acrylate or methacrylate.

11. The process of claim 1 wherein the acrylic monomer is selected from the group consisting of 2-ethylhexyl acrylate, n-butyl acrylate, 2-ethylhexyl methacrylate, hexyl methacrylate, n-butyl methacrylate, isobornyl methacrylate, and mixtures thereof.

12. The process of claim 1 wherein the allylic alcohol has the general structure: 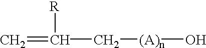
in which R is hydrogen, a C₁-C₁, alkyl, or a C₆-C₁₂ aryl group; A is an oxyalkylene group; and n, which is an average number of oxyalkylene groups, is within the range of 0 to about 15.

13. The process of claim 12 wherein n is within the range of about 1 to about 5.

14. The process of claim 12 wherein n is within the range of about 1 to about 2.

15. The process of claim 12 wherein the allylic alcohol is allyl alcohol monopropoxylate.

16. A process for making an acrylic polyol, said process being performed at reflux temperature under atmospheric pressure, essentially in the absence of styrene and in the absence of methyl acrylate and methyl methacrylate, and comprising:
(a) initially charging a reactor with an allylic alcohol, 0-75% of the total amount to be used of C₂ to C₂₀ alkyl or aryl acrylate or methacrylate and 0-100% of the total amount to be used of a free-radical initiator;
(b) heating the reactor contents to reflux; and
(c) gradually adding to the reactor the remaining acrylic monomer and initiator;
wherein the acrylic monomer has a boiling point the same as or higher than the allylic alcohol.

17. The process of claim 16 giving a total monomer conversion greater than about 90%.

18. The process of claim 17 wherein the free-radical initiator contains less than 30 wt % of water.
